(19) United States
(12) Patent Application Publication
(10) Pub. No.: US 2002/0161018 A1
(43) Pub. Date: Oct. 31, 2002

(54) Enhancement of the potency and efficacy of local anesthetics with ibutilide and other methanesulfonamide drugs

(76) Inventors: Forrest L. Smith, Richmond, VA (US); Raymond Lindsay, Charlottesville, VA (US)

Correspondence Address: WHITHAM, CURTIS & CHRISTOFFERSON, P.C., 11491 SUNSET HILLS ROAD, SUITE 340, RESTON, VA, 20190, US

(21) Appl. No.: 10/078,658

(22) Filed: Feb. 21, 2002

Related U.S. Application Data
(60) Provisional application No. 60/270,608, filed on Feb. 23, 2001.

(51) Int. Cl. A61K 31/46; A01N 37/18; A61K 31/16

(52) U.S. Cl. 514/304; 514/630; 514/601

(57) ABSTRACT

The efficacy of amide- and ester-linked local anesthetics is increased by administering methanesulfonamide compounds (for example, ibutilide and sotalol) as adjuvants with the local anesthetics. The effectiveness of these compounds as adjuvants is further increased by the concomitant administration of epinephrine. Pharmaceutical compositions comprising an amide- and ester-linked local anesthetic and a methanesulfonamide compound, which may further include epinepherine, are also provided.

This application claims priority of U.S. provisional application serial No. 60/270,608 filed Feb. 23, 2001, the complete contents of which is incorporated herein by reference.

This invention was made using the funds from the grants from the National institutes of Health having grants DA-01647. The government may have certain rights in the invention.

### BACKGROUND OF THE INVENTION

1. Field of the Invention

The invention generally relates to adjuvants for local anesthetics. In particular, the invention provides methods and compositions for administering methanesulfonamide compounds with ester- or amide-linked local anesthetics in order to increase the efficacy of the local anesthetics.

2. Background of the Invention

While local anesthesia is a necessary and widely used component of surgical procedures, it is not without associated disadvantages. Most local anesthetics have a relatively low therapeutic index that limits the amount of anesthetic that can be safely administered to provide effective nerve conduction block. Anaesthesiologists and nurse anesthetists recognize the potential of anesthetic toxicity to be a major life-threatening event. Toxicity can occur from inadvertent intravenous injection, inadvertent intrathecal injection during epidural administration, and from other inadvertent misapplications. In some cases, it is simply necessary to inject very large volumes—and consequently very high doses—of local anaesthetic in order to provide an adequate level of block. Subsequent absorption of these high doses has been known to lead to systemic anaesthetic shock.

In particular, local anesthetics are widely used in pediatric patients to provide regional nerve block anesthesia, or cutaneous anesthesia following subcutaneous or intradermal infiltration. Yet, a number of reports indicate that infants and children are more sensitive than adults to the toxic effects of local anesthetics (Berde et al. 1993; Ryan et al., 1993; Yan and Newman 1998; Malamed 1999; Verts 1999; Moore and Hersh, 2000). Like adults, children can experience CNS and cardiovascular toxicity resulting from high plasma levels of local anesthetic. Yet, infants and children have a lower seizure threshold that accounts for the high incidence of febrile seizures (Ryan et al., 1993). Infants in two reports and a 13-year old in another report experienced seizures following subcutaneous infiltration of lidocaine or bupivacaine (Ryan et al., 1993; Yan and Newman 1998; Jonville et al., 1990). The 13-year old also experienced ventricular fibrillation from which she recovered. The risk factors for both CNS and cardiovascular toxicity increases in sick children experiencing hypercarbia from respiratory acidosis. Under acidic conditions, local anesthetics shift from the unionized to the ionized form, thereby increasing the concentration of the drug moiety (ionized form) that affects nerves and cardiovascular tissue (Ryan et al., 1993). Clinicians could use much safer local anesthetic concentrations if including an adiuvant improved the potency and efficacy of the local anesthetic. In addition, much lower total body doses (mg/kg) of local anesthetic could be used to provide safe and effective anesthesia.

In addition, pain control in the dental treatment of children is very important. Yet studies examining the effectiveness of local anesthetics have revealed a high incidence of ineffective pain control. Observation of 361 children and 17 dentists specialized in pediatric dentistry revealed an 11.6% incidence of inadequate pain control (Nakai et al., 2000). Of these dentists, 88.2% were observed to have at least one patient experiencing pain after anesthetic treatment. The authors speculate that the incidence of inadequate pain control in children may be much greater in general practice. Again the inclusion of an adjuvant to improve the potency and efficacy of local anesthetics would be highly beneficial.

Epinephrine, which causes localized vasoconstriction, is often added to local anesthetics as an adjuvant. Epinephrine acts as a “chemical tourniquet”, slowing the redistribution of anesthetic from the area and thus prolonging the duration of action of the anesthetic. However, epinephrine itself can also produce toxic side effects, especially if inadvertent intravenous injection occurs.

It would be of great benefit to have available additional adjuvants for use with local anaesthetics in order to enhance their efficacy and potency. Such adjuvants could greatly increase the safety of administering local anesthetics by decreasing the amount required in order to achieve a desired effect. It would be especially advantageous if the adjuvants themselves were relatively non-toxic.

### SUMMARY OF THE INVENTION

The present invention provides methods for increasing the efficacy of local anesthetics by administering an adjuvant with the local anesthetic. The adjuvants are methanesulfonamide compounds which are currently utilized as Class III antiarrhythmic agents, examples of which include ibutilide and sotalol. The methanesulfonamide compounds are administered with a local anesthetic (e.g. an amide- or ester-linked local anesthetic such as bupivacaine) and a vasocontrictive substance such as epinephrine may also be administered with the local anesthethic and the methanesulfonamide.

The present invention also provides a method of providing local anesthesia to a patient in need thereof. The method comprises administering with an amide- or ester-linked local anesthetic a methanesulfonamide compound such as ibutilide and sotalol. The method may further include administration of a vasoconstrictive substance such as epinephrine.

The present invention also provides a pharmaceutical preparation comprising a local anesthetic and a methanesulfonamide compound. The composition may further comprise a vasoconstrictive substance such as epinephrine.

### BRIEF DESCRIPTION OF THE DRAWINGS

FIG. 1A-E. Representative methanesulfonamide compounds. A, Generic methanesulfonamide moiety; B, Ibutilide:, (±) N-[4-[4-(Ethylheptylamino)-1-hydroxybutyl] phenyl]methanesulfonamide (E)-2-butenedioate (2:1) salt; C, sotalol HCl: N-[4-[1-hydroxy-2-[(1-methylethyl)amino] ethyl]phenyl]methanesulfonamide monohydrochloride); D, dofetilide: N-[4[2-[methyl[2-[4-[(methylsulfonyl)amino] phenoxy] ethyl] amino] ethyl] phenyl] methanesulfonamide; E, Sematilide, N-[2-(diethylamino (ethyl]-4-[(methylsulfonyl) amino]benzamide monohydrochloride; E4031, [1-[2-(6-methyl-2-pyridyl)-ethyl]-4-(4-methylsulfonylaminobenzoyl)piperidine.

FIG. 2. Ibutilide enhances the local anesthetic effects of 0.5% bupivacaine. The plantar aspect of the hind-paw of male ICR mice was applied to the surface of a hot-plate device set at 56° C. to obtain the baseline paw-withdrawal latency. Vehicle, 0.5% bupivacaine, ibutilide (3.9 nmoles) or a mixture of these drugs was then injected in the popliteal space. The animals were then tested every 5 min for a period of 1-h. The data was analyzed by two-factor ANOVA followed by post hoc analysis by the Tukey's test. ◯=vehicle+vehicle; □=3.9 nmioles ibutilide+vehicle; ●=0.5% bupivacaine+vehicle; ▪=0.5% bupivacaine+3.9 nmoles ibutilide.

FIG. 3. Ibutilide enhances the local anesthetic potency of bupivacaine. Baseline hot-plate latencies were obtained by applying the plantar aspect of the hind-paw of male ICR mice on the surface of a hot-plate device set at 56° C. Vehicle or ibutilide (3.9 nmoles) was mixed with different percent bupivacaine solutions, and then injected in the popliteal space. The animals were tested 10-min later for construction of dose-response curves. ◯=vehicle; ●=3.9 nmoles ibutilide.

FIG. 4. Ibutilide enhances the local anesthetic effects of a low dose of bupivacaine with 1:200,000 epinephrine. Baseline hot-plate latencies were obtained before injecting vehicle or ibutilide (3.9 nmoles), 0.125% bupivacaine or a mixture of these drugs in the popliteal space. The animals were then tested every 5 min for a period of 1-h. The data was analyzed by two-factor ANOVA followed by post hoc analysis by the Tukey's test. ◯=vehicle+vehicle; □=3.9 nmoles ibutilide+vehicle; ●=0.125% bupivacaine+vehicle; ▪=0.125% bupivacaine+3.9 nmoles ibutilide.

FIG. 5. Ibutilide enhances the potency of bupivacaine with 1:200,000 epinephrine.

Baseline hot-plate latencies were obtained before injecting in the popliteal space with different percent bupivacaine+epinephrine solutions containing vehicle or ibutilide (3.9 nmoles). The animals were tested 10-min later for construction of dose-response curves.□=vehicle; ▪=3.9 nmoles ibutilide.

FIG. 6A and B. Sotalol enhances the anesthetic potency of bupivacaine. A, Baseline tail-flick latencies were obtained before infiltrating the tails of mice with 100 microL of 0.12% bupivacaine along with vehicle or increasing doses of sotalol. Thirty-min later, test latencies were obtained and the data was converted into percentage of maximum possible effect (% MPE) values. B, Baseline tail-flick latencies were obtained before infiltrating the tails of mice with 100 microL of vehicle (∘) or 10 nanomoles sotalol (▪) with increasing doses of bupivacaine. Thirty-min later, test latencies were obtained and the data was converted into percentage of maximum possible effect (%MPE) values.

### DETAILED DESCRIPTION OF THE PREFERRED EMBODIMENTS OF THE INVENTION

Applicants have made the surprising discovery that methanesulfonamides, when administered with local anesthetics (substances having an analgesic effect), significantly increase the potency of the anesthetics. As a result, the amount of local anesthetic that must be administered in order to achieve a desired degree of anesthesia can be reduced. The methanesulfonamide compounds themselves have never previously been shown to exhibit adjuvant anesthetic properties. Rather, they are members of the class of drugs known as Class III antiarrhythmic agents and are currently used for the treatment of heart disease. Because these compounds are already used for such medical purposes, many of them are already well-characterized, (for example, with respect to toxicity) and are already approved for clinical use in humans.

A further advantage of the present invention is that the amount of the methanesulfonamide compounds that must be administered with the anesthetic in order to achieve a desired effect is extremely low in comparison to a dose that would typically be utilized to treat arrhythmia, and is also well below the established toxicity levels of the drugs. Thus, the dose of methanesulfonamide resulting from normal redistribution away from the site of administration, or from an inadvertent injection of the adjuvant or an adjuvant/anesthetic mixture, would be well below that which would have an effect on the heart, or be toxic to the patient.

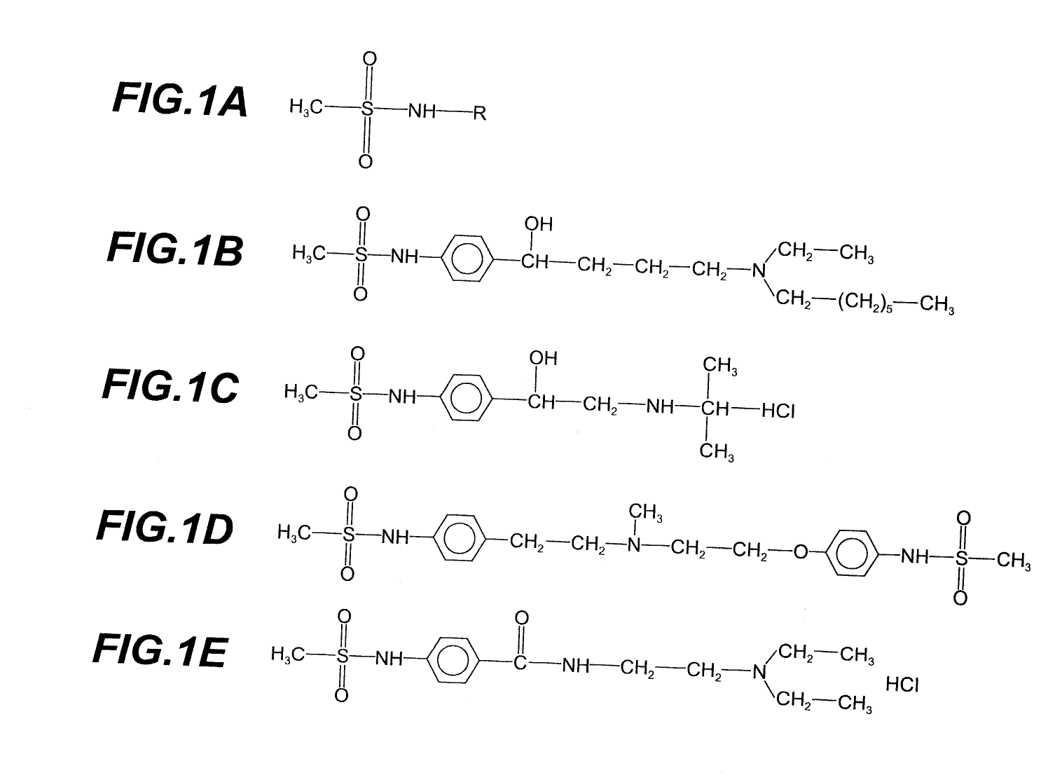

The types of compounds that may be utilized in the practice of the present invention are classified as methanesulfonamides. A generic methanesulfonamide compound is depicted in FIG. 1A, where R represents a variable constituent which can contain alkyls, aryls, amino, sulfyl, or other groups. Examples of members of this class of compound suitable for use in the practice of the present invention include but are not limited to: ibutilide (Corvert®, Pharmacia & Upjohn, Kalamzoo, Mich.) i.e. (±) -N-[4-[4-(Ethylheptylamino)-1-hydroxybutyl]phenyl] methanesulfonamide (E)-2-butenedioate (2:1) salt, see FIG. 1B; dofetilide (Pfizer US Pharmaceutical Group, New York N.Y.), i.e. N-[4[2-[methyl[2-[4-[(methylsulfonyl)amino] phenoxy]ethyl]amino]ethyl]phenyl] methanesulfonamide, see FIG. 1D; Sematalide (Berlex Labortories, Inc., Wayne, N.J.) i.e. N-[2-(diethylamino (ethyl]-4-[(methylsulfonyl)-amino] benzamide monohydrochloride, see FIG. 1E; sotalol HCl (Betapace®, Berlex Labortories, Inc., Wayne, N.J.) i.e. N-[4-[1-hydroxy-2-[(1-methylethyl) amino] ethyl]phenyl] methanesulfonamide monohydrochloride), see FIG. 1B; E4031 i.e. [1-[2-(6-methyl-2-pyridyl)-ethyl]-4-(4-methylsulfonylaminobenzoyl)piperidine].

In a preferred embodiment of the present invention, the methanesulfonamide compound that is utilized is ibutilide. In yet another preferred embodiment, the methanesulfonamide compound is sotalol. Distinguishing characteristics of these and other methanesulfonamide compounds are as follows: Sotalol is a Vaughan Williams Class III antiarrhythmic agent, is FDA-approved for the maintenance of normal sinus rhythm (NSR) in patients with symptomatic atrial fibrillation/atrial flutter (AFib/AFI) who are currently in NSR. Sotalol has both beta-adrenoreceptor blocking (Vaughan Williams Class II) and cardiac action potential duration prolongation (Vaughan Williams Class III) antiarrhythmic properties. Sotalol has been shown to prolong the plateau phase of cardiac action potential in isolated myocytes, as well as in isolated tissue preparations of ventricular or atrial muscle (Class III activity). Several mechanisms of action for sotalol have been proposed. In guinea pig ventricle, d-sotalol depressed the time-dependent delayed outward rectifying potassium current (IKr) (I=current; K=potassium; r=rectifying) (Malecot and Argibay, 1999). There seems to be little or no effect of sotalol on the slowly activating potassium current (IKs). Dofetilide appears to act very much like sotalol, while the other methanesulfonamide azimilide blocks both IKr and IKs channels.

Ibutilide appears to prolong repolarization in the heart by enhancing an inward depolarizing slow Na⁺ current (Naccarelli et al., 1996), also known as the “late inward Na⁺ current” (Wood et al., 2000). Blocking the “late inward Na⁺ current” causes an increase in the effective refractory period in the heart by prolonging the action potential duration. In addition, it has also been posited that ibutilide, like sotalol, affects the rectifying K⁺ current (IKr) (Wood et al., 2000). Thus, it appears that ibutilide and sotalol share a common mechanism of action of blocking IKr channels. Therefore, without being bound by theory, ibutilide and sotalol may enhance the potency of local anesthetics by blocking the IKr channel. Signficantly, peripheral nerves of the sympathetic nervous system appear to possess such IKr channels (Furukawa et al., 1999).

The practice of the present invention involves the administration of a methanesulfonamide compound with a local anesthetic. By “administration with” or “administered with” or “co-administered” we mean that the methanesulfonamide compound may be administered either concomitantly with the local anesthetic, or the methanesulfonamide may be administered in temporal juxtaposition to the anesthetic. For example, a single mixture containing both the methanesulfonamide and the anesthetic may be administered. Alternatively, the two moieties may be administered simultaneously from two different preparations, or one after the other, e.g. administration of the methanesulfonamide may be preceded or followed immediately or very soon (i.e. within a range of about 0 to about 60 minutes) by administration of the anesthetic. Alternatively, a mixture of the local anesthetic and methanesulfonamide may be formed and administered as a single formulation.

In a preferred embodiment of the invention, the local anesthetics are amide-linked or ester-linked anesthetics. Commercially available amide-linked anesthetics are derivatives of aniline, and are metabolized primarily in the liver by amidases. Other amide-linked anesthetics that are not derived from aniline may become commercially available at a later date. Examples of amide-linked local anesthetics that are appropriate for use in the instant invention include, but are not limited to, dibucaine, prilocaine, lidocaine, mepivacaine, bupivacaine, articaine, levobupivacaine, ropivacaine, tocanide and etidocaine. Also encompassing the scope of the instant invention are local anesthetic combinations such as prilocaine-lidocaine in EMLA® (i.e., Eutectic Mixture of Local Anesthetics) cream that is used for topical anesthesia, in which inclusion of a methanesulfonamide could increase the potency, efficacy or duration of action of EMLA®. Commercially available ester-linked anesthetics are derivatives of para-aminobenzoic acid, and are characteristically metabolized by hydrolysis of the ester linkage by plasma esterase, probably plasma cholinesterase. Other ester-linked anesthetics that are not derived from para-aminobenzoic acid may become commercially available at a later date. Examples of ester-linked local anesthetics which are appropriate for use in the instant invention include, but are not limited to, procaine, chloroprocaine, tetracaine, benzocaine, butamben picrate, and cocaine. However, those of skill in the art will recognize that other amide- and ester-linked local anesthetics exist or may be developed which would also be suitable for use in the practice of the present invention. All such suitable amide- and ester-linked local anesthetics are intended to be encompassed in the scope of the instant invention.

The present invention also provides an improved local anesthetic pharmaceutical composition comprising at least one amide- or ester-linked local anesthetic and at least one methanesulfonamide compound. The amide- or ester linked anesthetics include but are not limited to, for example, (amide-linked) dibucaine, prilocaine, lidocaine, mepivacaine, bupivacaine, articaine, levobupivacaine, ropivacaine, tocanide and etidocaine, or any local anesthetic combinations such as EMLA®, or (ester-linked) procaine, chloroprocaine, tetracaine, benzocaine, butamben picrate, and cocaine. Suitable methanesulfonamide compounds for inclusion in the pharmaceutical composition include but are not limited to: ibutilide, sotalol, dofetilide, sematilide, E4031. In general, the amount of methanesulfonamide compound in the pharmaceutical composition will be in the range of femtomole (10⁻¹⁵ moles) to millimole (10⁻³ mole) doses per kilogram body weight. The concentration of methanesulfonamide may also be expressed in concentration units ranging from femtomolar (10⁻¹⁵ moles/Liter) to millimolar (10⁻³ moles/Liter). Further, those of skill in the art will recognize that more than one (i.e. a combination of) methanesulfonamides may also be administered.

In addition, the pharmaceutical composition of the present invention may also comprise vasoconstrictive substances such as, for example, epinephrine or levonordefrin. In general, epinephrine will be provided in the amount of about 1:50,000 to 1:200,000, and levonordefrin in the amount of about 1:20,000.

The form of such a composition may be any of those which are well known to those of skill in the art, examples of which include but are not limited to liquid solutions suitable for injection or for intravenous administration, creams or gels suitable, for example, for topical administration, and the like. The compositions of the present invention may be administered in any form suitable for the method of administration.

Further components of such compositions may include such substances as wetting agents, various stabilizers, buffering agents, preservatives, sterilizing agents, coloring agents, emulsifying agents, surfactants, flavoring agents (e.g. for dental applications), and the like.

Further, the anesthetic may be in the form of a free base, an acid addition salt, or a pH buffered ionic solution containing the aesthetic in the free base or ionized form of the local anesthetic.

The improved local anesthetic of the present invention can be administered in any of a variety of ways which are well-known to those of skill in the art. “Administration” includes the injection or application of local anesthetic and methanesulfonamide mixed together for co-administration. “Administration” includes the injection of local anesthetic and metbanesulfonamide separately, either in the same site, or the administration of local anesthetic in the site to be anesthetized and administration of the methanesulfonamide by any other route, such as intravenous, subcutaneous, intradermal, intranasal, intraperitoneal, intrathoracic, epidural, spinal, intra-articular, per os, transdermal, transmucosal, transrectal. “Administration” includes, but is not limited to, the injection of local anesthetic with methanesulfonamide through an injection needle connected directly to a syringe, a catheter, or a combination of needle-catheter-syringe for injecting local anesthetic by hand, mechanical pump, isometric compression device, or other mechanical infusion device. Another combination encompassing “administration” includes a catheter-syringe combination for injecting local anesthetic by hand, or mechanical pump, or infusion device for what is commonly used for spinal, epidural or intra-articular administration. “Administration” includes, but is not limited to, the application of local anesthetic with methanesulfonamide through an aerosol spray driven by propellant gasses, compressed air, or other metered-dose spray administration method for topical, intranasal, per os, or any other route used to apply anesthetics by spray. “Administration” includes, but is not limited to, the application of local anesthetic with methanesulfonamide through an adhesive “patch” to provide transdermal or transmucosal absorption of local anesthetic. For example, Duragesic® adhesive patches are used to provide continuous absorption of the opiate fentanyl through the skin of humans suffering from chronic pain. Another example includes Lidoderm® patches that release lidocaine for transdermal absorption, for example, in the treatment of post-herpetic neuralgia. Those of skill in the art will recognize that the local anesthetic of the present invention may be administered by any suitable means known or under development. Further, administration may be at any suitable site, i.e. at an site of a body where it is desired to elicit an analgesic effect.

The methods and improved local anesthetic of the present invention may be utilized in any of a wide variety of procedures which are well-known to those of skill in the art. Such procedures include but are not limited to infiltration anesthesia (i.e. field blocks), orthopedic surgery brachial plexus blocks, synovial blocks, intercostal blocks, during various types of dental procedures, and the like. The methods and local anesthetic of the present invention may be utilized in any suitable medical procedure where it is advisable to utilize a local anesthetic.

In a preferred embodiment of the present invention, the subjects to whom the local anesthetic plus methanesulfonamide adjuvant is administered are human subjects. However, those of skill in the art will recognize that the methods of the present invention are equally applicable for veterinary purposes.

### EXAMPLES

### Material and Methods for Examples 1 and 2

Animals: Male ICR mice (25-30 gm) were obtained from Harlan Sprague Dawley. The mice were housed in groups of four to six and kept on a 12-h light/12-h dark cycle. Mice were tested during the light cycle; and the original groupings were maintained (i.e., mice were not recombined into new groups or isolated) to decrease infighting and consequent stress that could confound the results.

Behavioral Procedures: Following the procedure of Leszczynska and Kau (1992), each mouse was placed on a ¼inch wire mesh screen, which was then inverted; a mouse with no motor defects walks in a characteristic circular pattern, grasping the mesh with each paw and then letting go. Mice that could not walk normally at baseline were not used for testing. Each mouse also had its paw to be injected placed on a Syscom Model 35D hot plate set at 55° C. to test for sensory block. Baseline paw withdrawal latencies ranged between 2- to 3-sec. After collecting baseline data, the mice were injected with vehicle, ibutilide, or bupivacaine mixed with vehicle or ibutilide. At the appropriate times, motor scores and test paw withdrawal latencies were obtained. A 20-sec cut-off was employed to prevent tissue damage on the hot-plate test.

Injection Procedures: Each mouse was injected with drug solution using a 30 gauge,½ inch needle attached to a 1 ml syringe (needle and syringe from Becton Dickinson). The animals were gently restrained, while the limb to be injected was extended to expose the popliteal space, which is behind the knee of the mouse. The needle was inserted in the popliteal space, and 0.05 ml of drug solutions injected. The nerve bundle of the sciatic nerve passes through the popliteal space. Thus, the limb and paw distal to the injection is effectively anesthetized.

The motor and sensory tests were then repeated at 5-min intervals for a 1-h period. Mice that demonstrated complete motor block could not place their paw on the screen; the leg characteristically drooped when the screen was inverted. Some mice could place their paw on the screen, but could not grasp the screen with their digits; this was determined to be an intermediate phase. The motor block was determined to be resolved when the mouse could walk on the inverted screen using both the leg and paw. Sensory blockade was assessed by measuring in seconds, the time the bupivacaine-injected paw remained on hot-plate surface before withdrawing it. The test was terminated if the cut-off of 20-sec occurred during the test procedure. For time-course studies, two-factor repeated measures analysis of variance followed by the post hoc Tukey's test was used to assess for significant increases in paw withdrawal latencies compared to baseline. In addition, the effect of ibutilide to enhance bupivacaine was assessed, and compared with the appropriate controls at each time point during the 60-min test period. For dose-response curves, the data was converted to the percentage of maximum possible effect, which was calculated as: % MPE=[(Test−Baseline)/(20 −Baseline)]×100. ED₅₀ values were calculated using least-squares linear regression analysis followed by calculation of 95% confidence limits by the method of Bliss (1967). The absence of overlapping confidence limits was used to indicate significant differences in ED50 values between dose-response curves. Potency-ratio values with 95% confidence limits were calculated by the method of Colquhoun (1971).

Drugs: Sterile isotonic saline was used to dissolve and dilute drug solutions. Bupivacaine HCl was purchased from the MCV Hospitals Pharmacy (Winthrop Pharmaceuticals, NY, N.Y. ). To assure consistency, the same vial of bupivacaine was used for all injections involving bupivicaine. The concentration was varied by either diluting with normal saline, or by freeze-drying and then reconstituting the material with normal saline. The same vial of bupivacaine with 1:200,000 epinephrine was used for the second set of experiments. The epinephrine dose was adjusted by adding epinephrine to maintain a constant concentration of epinephrine 2.76×10⁻⁵ M. Ibutilide fumarate was purchased from the MCV Hospitals Pharmacy (Pharmacia & Upjohn Company of Kalamazoo, Mich.). The concentration of ibutilide was held constant at 3.9 nanomoles (nmoles) per mouse.

### Materials and Methods for Example 3

Methods of handling mice. Male ICR mice (Harlan Laboratories, Indianapolis, Ind.) weighing 27.0±0.4 g were housed 6 to a cage in animal care quarters maintained at 22±2 ° C. on a 12-h light-dark cycle. Food and water were available ad libitum. The mice were brought to a test room (22±2° C. , 12-h light-dark cycle), marked for identification and allowed 16-h to recover from transport and handling. The Institutional Animal Care and Use Committee at the Medical College of Virginia Campus of Virginia Commonwealth University approved all the procedures in this study.

The tail-flick test. The tail-flick test used to assess for infiltration anesthesia was developed by D'Amour and Smith (1941) and modified by Dewey et al (1970). The tail-flick device is designed to focus a light beam onto the site infiltrated with local anesthetic, without exposing non-injected tissue to the noxious radiant heat stimulus. The intensity was adjusted to yield baseline latencies of between 2- to 4-sec. A single baseline latency was obtained approximately 15-min before the mice were injected with drug. A 10-sec cut-off was used to prevent any potential tissue damage. In experiments testing for potential underlying infiltration anesthesia that was not observed with the higher heat stimulus, the intensity was reduced to yield baseline latencies of about 6-sec. A 12-sec cut-off was used in these experiments.

Subcutaneous infiltration of bupivacaine. Before infiltration of vehicle or drug, baseline tail-flick latencies were obtained by exposing the tail to noxious radiant heat at the site to be infiltrated with drug. The baseline average over the entire study was 3.1±0.1 sec. The mice were then restrained to allow us unencumbered access to the tail. A 26-gauge ⅝ needle was inserted approximately 1.0 to 1.3 cm longitudinally under the skin in the subcutaneous space in the dorsal aspect of the tail. The needle was attached to a 1-ml syringe (Becton Dickinson, Franklin Lakes, N.J. ) pre-filled with drug solution. A 100 ml volume of drug solution was infiltrated, and the needle was left in place for 5-s to prevent leakage of injectate from the site after removing the needle. During injection, the solution dispersed around the circumference of the tail in a rostral-caudal direction of about 2- to 3-cm. A slight blanching of the skin that reversed within 1 to 2 min indicated the extent of the spread of solution. A non-toxic marking pen was used to mark the most rostral spread of solution. Tail-flick testing was conducted below the area marked on the tail. These methods are detailed in a previous report (Smith et al. 1997).

Experimental design. Sotalol was solubilized and then mixed with bupivacaine HCl for co-infiltration of the tail. The influence of increasing concentrations of sotalol on the ED₅₀ dose of bupivacaine was also examined. Both baseline and test tail-flick latencies from each animal were converted into the percentage of maximum possible effect (% MPE) according to the method of Harris and Pierson (1964), which was calculated as: % MPE=[(test−baseline)/(10−baseline)]×100. The ordinate was % MPE, while the abscissa was expressed as the dose of sotalol. Complete bupivacaine dose-response curves were generated in the absence and presence of sotalol (10 nmole) for calculation of ED₅₀ and potency ratio values. The ordinate was % MPE, while the abscissa was expressed as the % bupivacaine dose.

Statistical analyses: The Tail-Flick Latency (sec) values were analyzed with one factor ANOVA for experiments in which different concentrations of sotalol were tested with an ED₅₀ dose of bupivacaine. A statistically significant F-value led to post hoc comparisons using the Tukey's test. The % MPE values for dose-response curves were analyzed in the following manner. ED₅₀ values were calculated using least squares linear regression analysis of % MPE values followed by calculation of 95% confidence limits (C.L.) according to the method of Bliss (1967). Dose-response curves were considered significantly different if the 95% C.L.s did not overlap. Tests for parallelism were conducted before calculation of potency ratio values and 95% confidence limits by the method of Colquhoun (1971). A potency-ratio value of greater than one, with a lower 95% confidence limit greater than one, was considered a significant difference in potency.

Drugs. Bupivacaine HCl was purchased from Sigma-Alrich, St. Louis, Mo. When necessary, dilutions of bupivacaine were made with sterile isotonic saline to achieve the desired final percent solution (Baxter Healthcare Corp., Deerfield, Ill). Sotalol HCl (Sigma-Alrich) was dissolved in sterile isotonic saline. The vehicle control consisted of isotonic saline. Sotolol HCl (Betapaceg®) was obtained from Sigma-Aldrich, St. Louis, Mo.

### RESULTS

### EXAMPLE 1

Enhancement of Bupivacaine HCl with Ibutilide. Experiments were conducted to determine whether ibutilide would enhance the local anesthetic effects of bupivacaine. Baseline hot-plate latencies were obtained by applying the plantar aspect of the hind-paw of male ICR mice on the surface of a hot-plate device set at 56° C. Vehicle or ibutilide (3.9 nmoles) was mixed with different percent bupivacaine, and then injected in the popliteal space (behind the mouse knee) around nerve fibers of the sciatic nerve. When an area below the knee is anesthetized in this manner, the procedure is commonly referred to as a “nerve block” or “peri-neural block”, or a “peri-sciatic block” procedure. This is a common procedure used in humans. The animals were tested 10-min later for construction of dose-response curves.

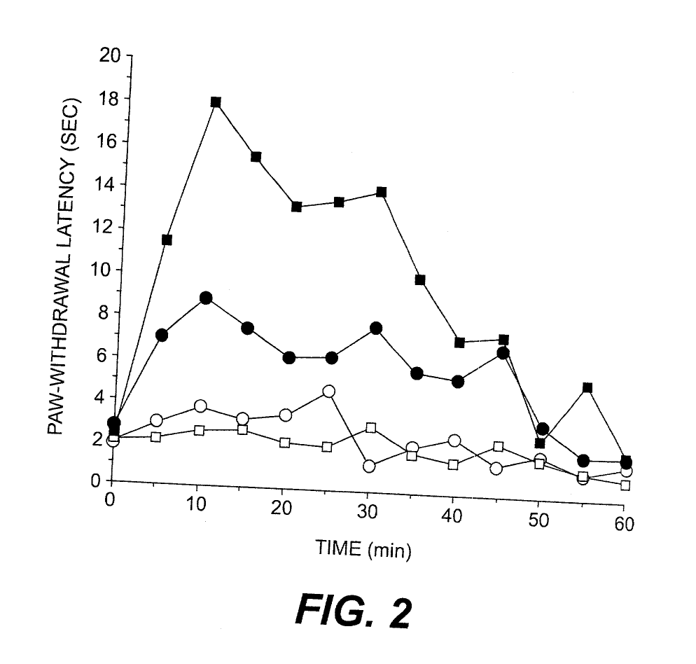

Time-course experiments reveal that 0.5% bupivacaine elicited a mild level of anesthesia that was significant at 10-min (FIG. 2). Mice injected with ibutilide (3.9 nmoles) alone exhibited no anesthesia throughout the 60-min test period. However, ibutilide markedly enhanced the local anesthetic effects of 0.5% bupivacaine for 35-min.

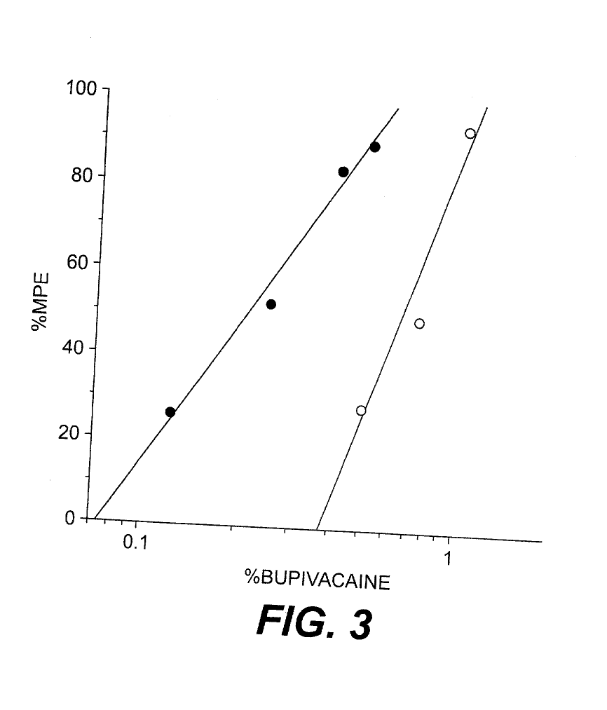

We next examined the effects of ibutilide on the potency of bupivacaine. As seen in FIG. 3, bupivacaine elicited dose-dependent anesthesia. However, in the presence of ibutilide the potency of bupivacaine was increased 2.8-fold (Table 1).

TABLE 1
Influence of Ibutilide on the local anesthetic potency of bupivacaine.

| | % Bupivacaine | Potency ratio |
|---|---|---|
| Treatment | ED50 (95% C.L.) | (95% C.L.) |
| Vehicle | 0.67% (0.59 to 0.77) | — |
| Ibutilide (3.9 nmoles) | 0.24% (0.19 to 0.30)** | 2.8 (1.9 to 3.8)** |

**Significantly different from bupivacaine + vehicle control

The results of FIG. 2 indicate that ibutilide alone as an adjuvant was inactive in eliciting anesthesia. However, ibutilide significantly increased the amount (or “efficacy”) of bupivacaine anesthesia, without increasing bupivacaine's duration of action. The results of FIG. 3 and Table 1 demonstrate that ibutilide significantly enhanced the “relative potency” of bupivacaine, indicated by a significant leftward shift in the dose-response curve with respect to the dose-response curve of mice injected with vehicle and increasing doses of bupivacaine. Thus, co-administration of the methanesulfonamide compound ibutilide significantly increased the “efficacy” and “relative potency” of the amide-linked local anesthetic bupivacaine in a typical “nerve block”, “peri-neural block”, or a “peri-sciatic block” procedure.

### EXAMPLE 2

Enhancement of Bupivacaine HCI+1:200,000 Epinephrine with Ibutilide. Experiments were conducted to assess whether the enhancement of bupivacaine anesthesia with ibutilide would be further increased by the inclusion of the vasocontrictor epinephrine. Baseline hot-plate latencies were obtained by applying the plantar aspect of the hind-paw of male ICR mice on the surface of a hot-plate device set at 56° C. For time-course studies vehicle or ibutilide (3.9 nmoles) was mixed with 0.125% bupivacaine+1:200,000 epinephrine solutions, and then injected in the popliteal space.

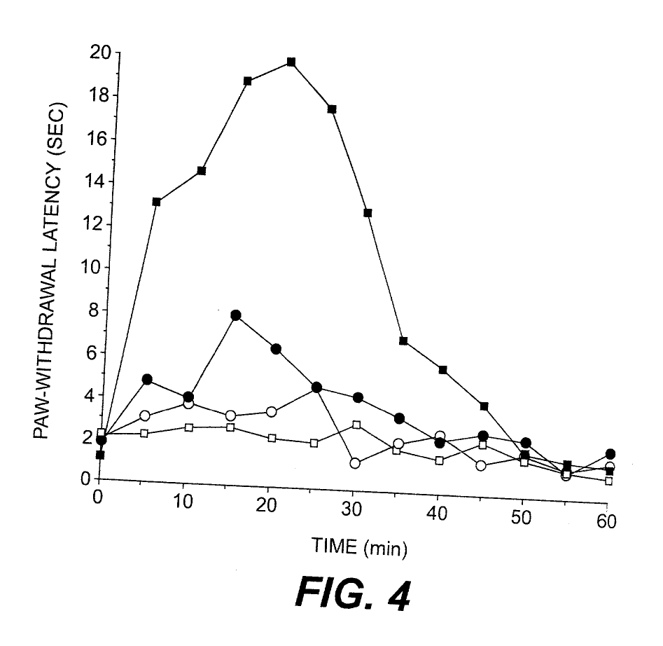

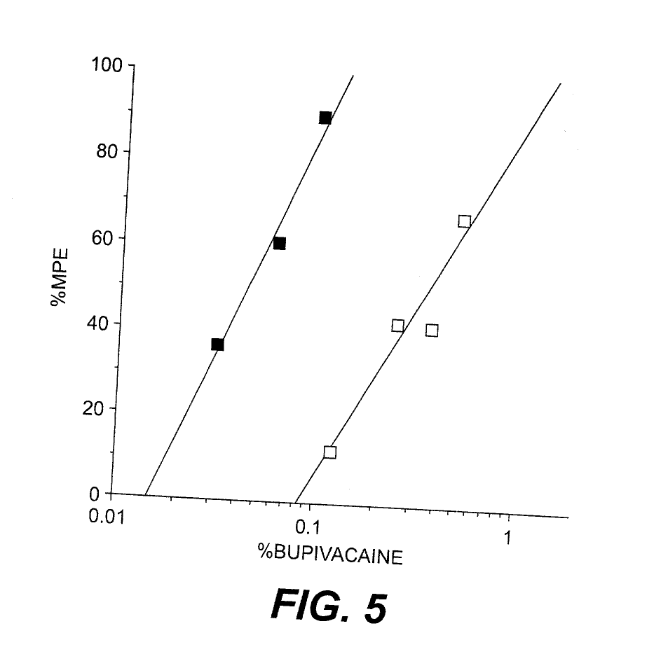

As seen in FIG. 4, 0.125% bupivacaine+1:200,000 epinephrine elicited local anesthesia that was significant at 15- and 20-min. Ibutilide in the presence of 1:200,000 epinephrine failed to elicit any anesthetic effects. However, ibutilide again greatly increased the amount (“efficacy”) of the 0.125% dose of bupivacaine. For dose-response curves, vehicle or ibultilde (3.9 nmoles) was mixed with increasing % bupivacaine solutions containing 1:200,000 epinephrine, and then injected in the popliteal space. The mice were tested 10-min later for construction of dose-response curves. When the potency of bupivacaine+1:200,000 epinephrine was examined in the absence and presence of ibutilide, it was found that ibutilide produced a dramatic 6.8-fold increase in the relative potency of bupivacaine+1:200,000 epinephrine (FIG. 5, Table 2).

TABLE 2
Ibultilide enhances the local anesthetic potency of bupivacaine
containing 1:200,000 epinephrine.

| | % Bupivacaine | Potency ratio |
|---|---|---|
| Treatment | ED50 (95% C.L.) | (95% C.L.) |
| Vehicle | 0.31% (0.22 to 0.44) | — |
| Ibutilide (3.9 nmoles) | 0.05% (0.04 to 0.06)** | 6.8 (4.3 to 12.7)** |

**Significantly different from bupivacaine + vehicle control

The time-course experiments demonstrate that including 1:200,000 epinephrine increased the “efficacy” of 0.125% bupivacaine. Epinephrine is a vasoconstrictor that slows the redistribution of bupivacaine from the injection site. Epinephrine may act to limit the redistribution of ibutilide as well. The dose-response curves demonstrate that 1:200,000 epinephrine also enhanced the ability of ibutilide to increase the relative potency of bupivacaine, possibly by slowing the re-distribution of bupivacaine and ibutilide.

### EXAMPLE 3

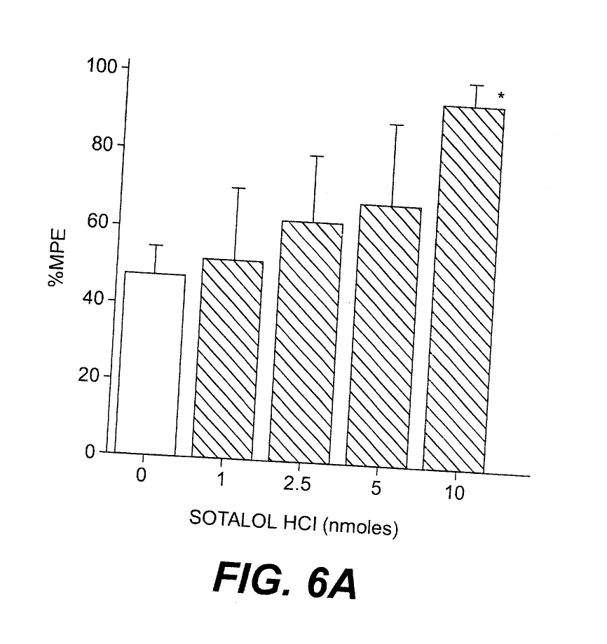

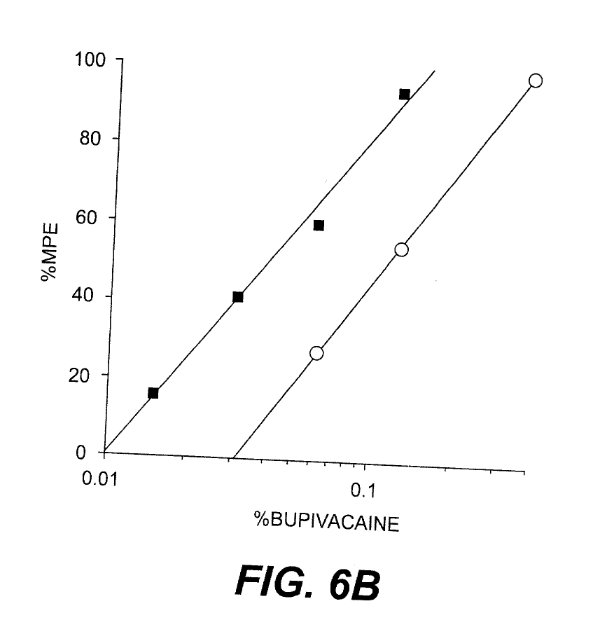

Enhancement of the efficacy of bupivacaine infiltration anesthesia with sotalol. Experiments were conducted to determine whether another methansulfonamide, sotalol, would enhance the anesthetic effects of bupivacaine. In this case, however, adminstration was via infiltration into the tails of mice. This type of “infiltration anesthesia” is commonly used in humans to anesthetize free-nerve endings in the skin and subcutaneous space in preparation for procedures such as suturing lacerations, forming surgical incisions with a scalpel. Baseline tail-flick latencies were obtained before infiltrating vehicle or sotalol with bupivacaine in the mouse tail. The mice were tested in the radiant heat tail-flick test 30-min later. Experiments were conducted to determine whether increasing doses of sotalol would enhance the “efficacy” of an ED₅₀ dose of bupivacaine (0.12%). Sotalol alone was inactive, however, sotalol enhanced in a dose-dependent fashion the anesthetic effects of an ED₅₀ dose of bupivacaine (0.12%) (FIG. 6A).

This example demonstrates that a second methanesulfonamide compound, sotalol, when delivered via an alternative route (infiltration) also significantly increased the anesthetic potency of bupivacaine. Sotalol by itself, however, displayed no anesthetic effects, a trait that is characteristic of adjuvant agents.

### EXAMPLE 4

Enhancement of the potency of bupivacaine with sotalol. Experiments were conducted to determine whether sotalol would enhance the anesthetic potency of bupivacaine in mice. Baseline tail-flick latencies were obtained before infiltrating vehicle or sotalol (10 nmoles) mixed with increasing % bupivacaine concentrations in the mouse tail for construction of bupivacaine dose-response curves. The animals were tested 30-min later in the radiant heat tail-flick test. The results indicate that sotalol significantly increased the potency of bupivacaine by 2.7-fold (FIG. 6B, Table 3).

TABLE 3
Influence of sotalol to enhance the local
anesthetic potency of bupivacaine.

| | % Bupivacaine | Potency ratio |
|---|---|---|
| Treatment | ED50 (95% C.L.) | (95% C.L.) |
| Vehicle | 0.11% (0.08 to 0.15) | — |
| Sotalol (10 nmoles) | 0.04% (0.03 to 0.06)** | 2.7 (1.6 to 4.5) |

**Significantly different from bupivacaine + vehicle control

This example demonstrates that the methanesulfonamide drug sotalol increased the relative potency of the amide-linked anesthetic bupivacaine by 2.7-fold. We note that this level of enhancement is nearly identical to the enhancement of bupivacaine's potency by ibutilide.

Conclusions: Different methanesulfonamide compounds have been shown to increase the efficacy of the local anesthetic bupivacaine, demonstrating that the capability of methanesulfonamide compounds to act as adjuvants with local anesthetics is a general phenomenon. Most noteworthy is the fact that ibutilide and sotalol had this effect when mice were injected by two different routes. In the ibutilide study, ibutilide and bupivacaine were given in the popliteal region (behind the mouse knee) around nerve fibers of the sciatic, simulating a “nerve block” or “peri-neural block”, or a “peri-sciatic block” procedure that is commonly used in humans. However, in the sotalol study, sotalol and bupivacaine were infiltrated just under the skin in the tail. This is commonly called “infiltration anesthesia”, and is used to anesthetize free-nerve endings in the skin and subcutaneous space. This type of anesthesia is commonly used for suturing lacerations in humans, or is infiltrated into an area to be incised with a scalpel for surgery. Thus, in one case the nerve fiber bundles were anesthetized so the whole limb of the mouse was numb (nerve block), and in the other case, the free nerve endings in the skin were anesthetized so that the area of numbness in the mouse tail was limited by the spread of anesthetic under the skin (infiltration anesthesia). These results suggest that methanesulfonamide compounds such as ibutilide and sotalol will enhance the potency of local anesthetics, regardless of how the local anesthetic is injected. The potency of ibutilide was enhanced further (i.e., 6.8-fold) when epinephrine was included. We anticipate that the sotalol/bupivacaine combination will be similarly enhanced by the addition of epinephrine.

These findings are especially important when one considers the current commercially used concentrations of bupivacaine, typically 0.25%, 0.5% and 0.75%. In general, the highest concentration utilized is 0.5%; 0.75% is rarely used because of the fear of toxicity. However, a methanesulfonamide compound such as ibutilide or sotalol is co-administered, given the fact that they cause a 2.8-fold increase in potency, 0.18% bupivacaine (0.5%/2.8-fold=0.18%) would be just as potent as 0.5% bupivacaine.

Likewise, if a methansulfonamide compound such as ibutilide or sotalol plus epinephrine is co-administered, only 0.07% bupivacaine would be just as potent as 0.5% bupivacaine, given the 6.8 fold increase in potency observed with this combination (0.5%/6.8-fold=0.07%). Inadvertent intravenous administration of 0.18% or 0.07% bupivacaine would not result in systemic toxicity or fatality, which is currently the greatest concern of anesthesiologista, surgeons, and dentists.

Similarly, when one considers the issue of total dose, the current accepted maximum dose of bupivacaine is 2 mg/kg. For a 70 kg (i.e. 154 lb) person, 140 mg of bupivacaine would be required. Unfortunately, the administration of amounts of local anesthetics greater than about 100 mg generates concern due to potential toxicity. However, if a methanesulfonamide adjuvant is co-administered, the required amount to achieve the same effect would be decreased to 50 mg (140 mg/2.8-fold=50 mg). The increase is even more dramatic (reduction to 21 mg) if epinephrine is also co-administered (140 mg/6.8-fold=21 mg). Total doses in the range of 21 or 50 mg are very reasonable. The danger of toxicity or fatality due, for example, to inadvertent administration, would decrease substantially, greatly enhancing the safety of local anesthetics.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

### REFERENCES

Berde C B. Toxicity of local anesthetics in infants and children. J Pediatr. 1993 May;122(5 Pt 2):S14-20.

Bliss, C. I., Statistics in Biology, p. 439. McGraw-Hill, New York, 1967.

Colquhoun, D., Lectures on biostatistics: An introduction to statistics with applications in biology and medicine, Oxford, Clarendon Press, 1971, pp. 327-33

D'Amour F E, Smith D L: A method of determining loss of pain sensation. J Pharmacol Exp Ther 1941; 72: 74-79

Dewey W L, Harris L S, Howes J F, Nuite J A: The effect of various neurohumoral modulators on the activity of morphine and the narcotic antagonists in the tail-flick and phenylquinone tests. J Pharmacol Exp Ther 1970; 175: 435-42

Foster R H, Wilde M I, Markham A: Ibutilide. A review of its pharmacological properties and clinical potential in the acute management of atrial flutter and fibrillation. Drugs. 1997 Aug;54(2):312-30.

Furukawa Y, Miyashita Y, Nakajima K, Hirose M, Kurogouchi F, Chiba S: Effects of verapamil, zatebradine, and E-4031 on the pacemaker location and rate in response to sympathetic stimulation in dog hearts. J Pharmacol Exp Ther. 1999 June;289(3):1334-42.

Harris L, Pierson A: Some narcotic antagonists in the benzomorphan series. J Pharmacol Exp Ther 1964; 143: 141-8

Jonville A P, Barbier P, Blond M H, Boscq M, Autret E, Breteau M. Accidental lidocaine overdosage in an infant. J Toxicol Clin Toxicol. 1990;28(1):101-6.

Leszczynska K, & Kau S:A Sciatic Nerve Blockade Method to Differentiate Drug-Induced Local Anesthesia from Neuromuscular Blockade in Mice. Journal of Pharmacological and Toxicological Methods 1992; 27:85-93.

Malamed S F. Morbidity, mortality and local anaesthesia. Prim Dent Care. 1999 January;6(1):11-5.

Moore P A, Hersh E V. Local anesthesia toxicity review revisited. Pediatr Dent. 2000 January-February;22(1):7-8.

Naccarelli G V, Lee K S, Gibson J K, VanderLugt J: Electrophysiology and pharmacology of ibutilide. Am J Cardiol. 1996 October 17;78(8A):12-6.

Nair L A, Grant A O. Emerging class III antiarrhythmic agents: mechanism of action and proarrhythmic potential. Cardiovasc Drugs Ther. 1997 April;11(2):149-67.

Nakai Y, Milgrom P, Mancl L, Coldwell S E, Domoto P K, Ramsay D S: Effectiveness of local anesthesia in pediatric dental practice. J Am Dent Assoc. 2000 December;131(12):1699-705.

Ryan C A, Robertson M, Coe J Y. Seizures due to lidocaine toxicity in a child during cardiac catheterization. Pediatr Cardiol. 1993 March.;14(2):116-8.

Smith F L: Regional cutaneous differences in the duration of bupivacaine local anesthesia in mice. Life Sci 1997; 60: 1613-21 Verts B E. Local anesthesia toxicity review. Pediatr Dent. 1999 September-October;21(6):375.

Wood M A, Fuller I, Hong A: Effects of sodium channel blockade on ibutilide induced prolongation of repolarization in the isolated rabbit ventricle. J Interv Card Electrophysiol. 2000 October;4(3):481-7.

Yan A C, Newman R D: Bupivacaine-induced seizures and ventricular fibrillation in a 13-year-old girl undergoing wound debridement. Pediatr Emerg Care. 1998 October;14(5):354-5.

We claim:

1. A method for increasing the potency of an amide-linked or ester-linked local anesthetic in a patient comprising the step of,
administering at least one methanesulfonamide compound with an amide-linked or ester-linked local anesthetic or combination thereof.

2. The method of claim 1 wherein said amide-linked local anesthetic is selected from the group consisting of dibucaine, articaine, levobupivacaine, ropivacaine, todocaine, lidocaine, prilocaine, mepivacaine, bupivacaine, and etidocaine.

3. The method of claim 1 wherein said ester-linked local anesthetic is selected from the group consisting of procaine, chloroprocaine, benzocaine, butamben, picrate, cocaine, and tetracaine.

4. The method of claim 1 further comprising the step of administering a vasoconstrictive substance to said patient.

5. The method of claim 4 wherein said vasoconstrictive substance is selected from the group consisting of epinephrine and levonordefrin.

6. The method of claim 1 wherein said at least one methanesulfonamide compound is selected from the group consisting of ibutilide, sotalol, E4031, dofetilide and sematilide.

7. A method of providing local anesthesia to a patient in need thereof, comprising the step of
administering to said patient at least one amide-linked or ester-linked local anesthetic together with at least one methanesulfonamide compound.

8. The method of claim 7 wherein said at least one amide-linked local anesthetic is selected from the group consisting of dibucaine, articaine, levobupivacaine, ropivacaine, todocaine, lidocaine, prilocaine, mepivacaine, bupivacaine, and etidocaine.

9. The method of claim 7 wherein said at least one ester-linked local anesthetic is selected from the group consisting of procaine, chloroprocaine, benzocaine, butamben, picrate, cocaine, and tetracaine.

10. The method of claim 7 further comprising the step of administering a vasoconstrictive substance to said patient.

11. The method of claim 10 wherein said vasoconstrictive substance is selected from the group consisting of epinephrine and levonordefrin.

12. The method of claim 7 wherein said at least one methanesulfonamide compound is selected from the group consisting of ibutilide, solatol, E4031, dofetilide and sematilide.

13. A pharmaceutical composition comprising,
at least one amide-linked or ester-linked local anesthetic, and
at least one methanesulfonamide compound.

14. The pharmaceutical composition of claim 13 further comprising a vasoconstrictive substance.

15. The method of claim 14 wherein said vasoconstrictive substance is selected from the group consisting of epinephrine and levonordefrin.

16. The pharmaceutical composition of claim 13 wherein said at least one amide-linked local anesthetic is selected from the group consisting of dibucaine, articaine, levobupivacaine, ropivacaine, todocaine, lidocaine, prilocaine, mepivacaine, bupivacaine, and etidocaine.

17. The pharmaceutical composition of claim 13 wherein said at least one ester-linked local anesthetic is selected from the group consisting of procaine, chloroprocaine, benzocaine, butamben, picrate, cocaine, and tetracaine.

18. The pharmaceutical composition of claim 13 wherein said at least one methanesulfonamide compound is selected from the group consisting of ibutilide, solatol, E4031, dofetilide and sematilide.
